(19) United States
(12) Patent Application Publication
(10) Pub. No.: US 2014/0234307 A1
(43) Pub. Date: Aug. 21, 2014

(54) METHOD OF TREATING MULTIPLE SCLEROSIS BY INTRATHECAL DEPLETION OF B CELLS AND BIOMARKERS TO SELECT PATIENTS WITH PROGRESSIVE MULTIPLE SCLEROSIS

(71) Applicant: The United States of America,as represented by the Secretary,Department of Health and Human Services, Bethesda, MD (US)

(72) Inventors: Bibiana Bielekova, Kensington, MD (US); Matthew L. Herman, Philadelphia, PA (US)

(21) Appl. No.: 14/346,473

(22) PCT Filed: Sep. 27, 2012
(86) PCT No.: PCT/US2012/057583

§ 371 Date: Mar. 21, 2014

Related U.S. Application Data
(60) Provisional application No. 61/539,870, filed on Sep. 27, 2011.

(51) Int. Cl. C07K 16/28; G01N 33/68; A61K 9/00

(52) U.S. Cl. CPC C07K 16/2887; A61K 9/0085; G01N 33/6869; G01N 33/6863
USPC 424/133.1; 435/7.92; 436/501

(57) ABSTRACT

Described herein are methods of treating multiple sclerosis (MS), such as secondary progressive MS (SPMS), by intrathecally administering a B cell depleting agent, such as rituximab, alone or in combination with intravenous administration of a B cell depleting agent. Also described is the use of IL-12p40, CXCL13, or both as CSF biomarkers for selecting a patient with progressive MS as a candidate for treatment with an intrathecal immunomodulatory therapy, and for identifying a progressive MS patient as having meningeal inflammation. The present disclosure also describes a method of evaluating the effectiveness of a therapy for treating progressive MS by measuring the level of IL-12p40, CXCL13, or both in the CSF of the patient before and after treatment. A decrease in the level of IL-12p40, CXCL13, or both after treatment indicates the therapy is effective for treating progressive MS.

### CROSS REFERENCE TO RELATED APPLICATIONS

This application claims the benefit of U.S. Provisional Application No. 61/539,870, filed Sep. 27, 2011, which is herein incorporated by reference in its entirety.

### FIELD

This disclosure concerns intrathecal (IT) administration of therapeutic agents, for example monoclonal antibodies, that deplete B cells from the intrathecal compartment for the treatment of multiple sclerosis (MS). This disclosure further concerns biomarkers for identifying MS patients with meningeal inflammation, and use of the biomarkers to evaluate and stratify patients for treatment.

### BACKGROUND

Multiple sclerosis (MS) is a chronic, neurological, autoimmune, demyelinating disease. MS can cause blurred vision, unilateral vision loss (optic neuritis), loss of balance, poor coordination, slurred speech, tremors, numbness, extreme fatigue, changes in intellectual function (such as memory and concentration), muscular weakness, paresthesias, and blindness. Many subjects develop chronic progressive disabilities, but long periods of clinical stability may interrupt periods of deterioration. Neurological deficits may be permanent or evanescent. In the United States there are about 250,000 to 400,000 persons with MS, and every week about 200 new cases are diagnosed. Worldwide, MS may affect 2.5 million individuals. Because it is not considered contagious, which would require U.S. physicians to report new cases, and because symptoms can be difficult to detect, the incidence of disease is only estimated and the actual number of persons with MS could be much higher.

The pathology of MS is characterized by an abnormal immune response directed against the central nervous system. In particular, T-lymphocytes are activated against the myelin sheath of the neurons of the central nervous system causing demyelination. In the demyelination process, myelin is destroyed and replaced by scars of hardened “sclerotic” tissue which is known as plaque. These lesions appear in scattered locations throughout the brain, optic nerve, and spinal cord. Demyelination interferes with conduction of nerve impulses, which produces the symptoms of multiple sclerosis. Most subjects recover clinically from individual bouts of demyelination, producing the classic remitting and exacerbating course of the most common form of the disease known as relapsing-remitting multiple sclerosis (RRMS). The majority of patients with RRMS will develop secondary-progressive multiple sclerosis (SPMS), characterized by progressive accumulation of neurological disability, despite lack of formation of focal inflammatory lesions that can be recognized on brain magnetic resonance imaging (MRI) as contrast-enhancing lesions (CELs). CELs are areas where the intravenously administered contrast agent can leak into the brain and spinal cord parenchyma due to the opening of the blood brain barrier (BBB). These are also the areas where therapeutic agents, including large molecules such as monoclonal antibodies, can gain access into the brain or spinal cord tissue. While therapies that effectively inhibit brain inflammation in RRMS have occasionally shown therapeutic efficacy in SPMS, this effect has always been limited to those patients who continue to have CELs. Thus, for SPMS patients who do not have CELs, and who therefore have no opening of the BBB, there are currently no effective disease-modifying treatments. Approximately 10% of MS patients develop primary progressive multiple sclerosis (PPMS), characterized by lack of CELs and clinically progressive accumulation of disability. Like for SPMS patients without CELs, there are currently no effective treatments for PPMS patients.

Rituximab is a genetically engineered chimeric monoclonal antibody that specifically binds CD20 used in the treatment of lymphoma, leukemia, transplant rejection and autoimmune disorders, including multiple sclerosis. Rituximab contains murine light and heavy chain variable regions and human gamma 1 heavy chain and kappa light chain constant regions. The chimeric antibody is composed of two heavy chains of 451 amino acids and two light chains of 213 amino acids and has an approximate molecular weight of 145 kD. Rituximab was genetically engineered using the murine 2B8 antibody (U.S. Pat. No. 6,455,043; U.S. Pat. No. 5,736,137).

### SUMMARY

Disclosed herein are methods for the treatment of MS by intrathecally administering an agent that promotes depletion of B cells from the intrathecal compartment. B cell depleting agents include, for example, monoclonal antibodies that bind B cell surface antigens, such as but not limited to CD19, CD20 and CD22. In particular examples herein, the B cell depleting agent is rituximab.

Provided herein is a method of treating MS by intrathecally administering a B cell depleting agent, such as a monoclonal antibody specific for a B cell surface antigen, alone or in combination with intravenous administration of the agent. In some embodiments, the method includes selecting a subject with MS and administering to the subject a therapeutically effective amount of the B cell depleting agent intrathecally, thereby treating the subject with MS. In some examples, the B cell surface antigen is CD20. In other examples, the B cell surface antigen is CD19 or CD22.

In some embodiments, provided herein is a method of treating MS by intrathecally administering rituximab, alone or in combination with intravenous administration of rituximab. In some embodiments, the method includes selecting a subject with MS and administering to the subject a therapeutically effective amount of rituximab intrathecally, thereby treating the subject with MS. In some examples, the method further includes administering a therapeutically effective amount of rituximab intravenously.

Also provided herein is a method of selecting a patient with progressive MS as a candidate for treatment with an intrathecal immunomodulatory therapy. In some embodiments, the method includes measuring the level of IL-12p40, CXCL13, or both in the cerebral spinal fluid (CSF) of the patient. An increase in the level of IL-12p40, CXCL13, or both relative to a control, indicates the subject is a candidate for treatment with an intrathecal immunomodulatory therapy. In some examples, the intrathecal immunomodulatory therapy includes intrathecal administration of rituximab.

Further provided is a method of identifying a progressive MS patient as having meningeal inflammation. In some embodiments, the method includes measuring the level of IL-12p40, CXCL13, or both in the CSF of the patient. An increase in the level of IL-12p40, CXCL13, or both relative to a control, indicates that the MS patient has meningeal inflammation.

The present disclosure also provides a method of evaluating the effectiveness of a therapy for treating progressive MS by measuring the level of IL-12p40, CXCL13, or both in the CSF of the patient before and after treatment. A decrease in the level of IL-12p40, CXCL13, or both after treatment indicates the therapy is effective for treating progressive MS.

The foregoing and other features and advantages will become more apparent from the following detailed description of several embodiments, which proceeds with reference to the accompanying figures.

### BRIEF DESCRIPTION OF THE FIGURES

FIG. 1 is a series of flow cytometry plots showing the effect of IT and intravenous (IV) rituximab treatment on B cell and T cell responses in MS patients. Patients were administered a single dose (25 mg) of IT rituximab and a first IV dose (200 mg) of rituximab. After two weeks, patient received a second dose (200 mg) of IV rituximab. B and T cell responses were evaluated before, immediately after and 3 months after treatment. IV rituximab treatment led to 99.5% depletion of B cells from the systemic circulation (page 1). The selected IT dose (25 mg) led to 85% depletion of B cells from cerebrospinal fluid 3 months after administration (page 2). The proportion of T cells in the cerebrospinal fluid with an in vivo activated phenotype (i.e. express HLA-DR) decreased 3 months after administration of IT rituximab by 82% for CD4+ T cells and by 70% for CD8+ T cells.

FIGS. 2A and 2B are graphs showing IL-12p40, CXCL13 and IL-8 CSF levels in patients with relapsing-remitting multiple sclerosis (RRMS), primary-progressive multiple sclerosis (PPMS), secondary-progressive multiple sclerosis (SPMS), all MS types (All MS), clinically isolated syndrome (CIS), other inflammatory neurological diseases (OIND), and non-inflammatory neurological diseases (NIND) in the pilot (A) and confirmatory (B) cohorts. The short black line is the median, and the lower detection limit is indicated by the dotted line. *P<0.05 Dunn's post test (DPT) on four groups, **P<0.05 DPT on six groups, ***P<0.05 DPT on both four and six groups.

FIG. 3 is a series of graphs showing levels of CXCL13 pre-treatment and 3-months post-treatment. Shown are the levels of CXCL13 in three patients treated with IT rituximab (top), two patients treated with placebo (bottom left and center), and in a cohort of ten patients treated with a different (non-rituximab) MS therapy (bottom right).

### DETAILED DESCRIPTION

### I. Abbreviations

BBB blood brain barrier

CIS clinically isolated syndrome

CEL contrast-enhancing lesion

CNS central nervous system

CSF cerebral spinal fluid

DMTh disease modifying therapies

ELISA enzyme-linked immunosorbent assay

HLA human leukocyte antigen

IL interleukin

IT intrathecal

IV intravenous

MRI magnetic resonance imaging

MS multiple sclerosis

NIND non-inflammatory neurological disease

OCB oligoclonal bands

OIND other inflammatory neurological disease

PPMS primary progressive multiple sclerosis

RRMS relapsing remitting multiple sclerosis

SPMS secondary progressive multiple sclerosis

### II. Terms and Methods

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8). In order to facilitate review of the various embodiments of the disclosure, the following explanations of specific terms are provided:

Administer: As used herein, administering a composition (e.g. an antibody, such as rituximab) to a subject means to give, apply or bring the composition into contact with the subject. Administration can be accomplished by any of a number of routes, such as, for example, intravenous, intrathecal, topical, oral, subcutaneous, intramuscular, intraperitoneal and intramuscular.

Antibody: Immunoglobulin molecules and immunologically active portions of immunoglobulin molecules, e.g., molecules that contain an antigen binding site that specifically binds (immunoreacts with) an antigen.

A naturally occurring antibody (e.g., IgG, IgM, IgD) includes four polypeptide chains, two heavy (H) chains and two light (L) chains interconnected by disulfide bonds. However, it has been shown that the antigen-binding function of an antibody can be performed by fragments of a naturally occurring antibody. Thus, these antigen-binding fragments are also intended to be designated by the term “antibody.” Specific, non-limiting examples of binding fragments encompassed within the term antibody include (i) a Fab fragment consisting of the V_{L}, V_{H}, C_{L} and C_{H1} domains; (ii) an F_{d} fragment consisting of the V_{H} and C_{H1} domains; (iii) an Fv fragment consisting of the V_{L} and V_{H} domains of a single arm of an antibody, (iv) a dAb fragment (Ward et al., Nature 341:544-546, 1989) which consists of a V_{H} domain; (v) an isolated complementarity determining region (CDR); and (vi) a F(ab′)₂ fragment, a bivalent fragment comprising two Fab fragments linked by a disulfide bridge at the hinge region.

Immunoglobulins and certain variants thereof are known and many have been prepared in recombinant cell culture (e.g., see U.S. Pat. Nos. 4,745,055 and 4,444,487; WO 88/03565; EP 256,654; EP 120,694; EP 125,023; Falkner et al., Nature 298:286, 1982; Morrison, J. Immunol. 123:793, 1979; Morrison et al., Ann. Rev. Immunol. 2:239, 1984).

B cell depleting agent: Any compound, such as a monoclonal antibody, that promotes a reduction in the number of B cells in a subject or in particular anatomical region of a subject (such as in the intrathecal compartment). “Depletion” of B cells need not be complete depletion, but encompasses any significant reduction in the number of B cells, such as a reduction of at least 10%, at least 20%, at least 30%, at least 40%, at least 50%, at least 60%, at least 70% or at least 80%. Thus, in some examples herein, a B cell depleting agent reduces the total number of B cells in a subject, such as within the intrathecal compartment of the subject. B cell depleting agents include, for example, monoclonal antibodies that target B cell surface antigens, such as but not limited to CD19, CD20 and CD22.

Control: A “control” refers to a sample or standard used for comparison with an experimental sample, such as a sample (e.g. a CSF sample) obtained from a patient with multiple sclerosis to be tested for protein biomarker levels (such as IL-12p40 or CXCL13). In some embodiments, the control is a sample obtained from a healthy patient. In other embodiments, the control is a historical control or reference standard (i.e. a previously tested control sample or group of samples that represent baseline or normal values, such as the level of IL-12p40 or CXCL13 expression in the CSF of a healthy subject). In other embodiments herein, the “control” is a patient that has been administered a placebo or a healthy control subject (i.e. a subject that does not have MS).

CD19: A protein expressed on the surface of follicular dendritic cells and B cells. In B cells, CD19 is expressed at the earliest stages of B cell development and on mature B cells. CD19 is found on both normal and transformed B cells.

CD19 monoclonal antibodies: Any monoclonal antibody, including human, mouse, chimeric or engineered antibodies, that specifically binds CD 19.

Exemplary anti-CD19 antibodies include BU-12 (Flavell et al., Br J Cancer 72(6):1373-1379, 1995) and huB4 (a humanized mouse monoclonal antibody, used in the SAR3419 immunoconjugate; Al-Katib et al., Clin Cancer Res 15(12):4038-4045, 2009).

CD20: The CD20 protein (cluster of differentiation 20, also called human B-lymphocyte-restricted differentiation antigen or Bp35) is a hydrophobic transmembrane protein with a molecular weight of approximately 35 kD located on pre-B and mature B lymphocytes (Valentine et al., J. Biol. Chem. 264(19):11282-11287, 1989; and Einfield et al., EMBO J. 7(3):711-717, 1988). In vivo, CD20 is found on the surface of greater than 90% of B cells from peripheral blood or lymphoid organs and is expressed during early pre-B cell development and remains expressed until plasma cell differentiation. CD20 is present on both normal B cells and malignant B cells, but is not found on hematopoietic stem cells, pro-B cells, normal plasma cells, or other normal tissues (Tedder et al., J. Immunol. 135(2):973-979, 1985). CD20 is involved in regulating early steps in the activation and differentiation process of B cells (Tedder et al., Eur. J. Immunol. 16:881-887, 1986) and can function as a calcium ion channel (Tedder et al., J. Cell. Biochem. 14D:195, 1990). The antibody rituximab specifically binds CD20.

CD20 monoclonal antibodies: Any monoclonal antibody, including human, mouse, chimeric or engineered antibodies, that specifically binds CD20. Exemplary anti-CD20 antibodies that have been evaluated in clinical studies, and in some cases approved for human use, include Ofatumumab (a human antibody; also known as ARZERRA™ and HuMax-CD20), ocrelizumab (a humanized antibody), veltuzumab (a humanized antibody), obinutuzumab (a humanized antibody; also known as GA101), AME-133v (an Fc-engineered humanized mAb), PRO131921 (a humanized antibody; also known as version 114 or v114) and LFB-R603/EMAB-6 (a chimeric mouse/human antibody). Anti-CD20 monoclonal antibodies that have been approved for clinical use in the United States, or are currently in clinical trials, are reviewed in Oflazoglu and Audoly, mAbs 2(1):14-19, 2010.

CD22: A protein found on the surface of mature B cells and some immature B cells. CD22 is a member of the immunoglobulin superfamily.

CD22 monoclonal antibodies: Any monoclonal antibody, including human, mouse, chimeric or engineered antibodies, that specifically binds CD22. An exemplary anti-CD22 antibody that has been evaluated in clinical studies is Epratuzumab, a humanized antibody (also known as LymphoCide).

Cerebral spinal fluid (CSF): A clear, colorless bodily fluid that occupies the subarachnoid space and the ventricular system around and inside the brain and spinal cord.

CXCL13 (chemokine (C—X—C motif) ligand 13): A CXC chemokine strongly expressed in the follicles of the spleen, lymph nodes, and Peyer's patches. It preferentially promotes the migration of B lymphocytes. CXCL13 is also known as BLC; BCA1; ANGIE; BCA-1; BLR1L; ANGIE2; and SCYB13. See NCBI Gene ID 10563 for human CXCL13.

Detecting expression of a gene: Determining the existence, in either a qualitative or quantitative manner, of a particular nucleic acid or protein product (such as IL-12p40 or CXCL13). Exemplary methods of detecting the level of protein expression include Western blot, immunohistochemistry, ELISA and mass spectrometry. Exemplary methods of detecting the level of nucleic acid (such as mRNA) include RT-PCR, Northern blot and in situ hybridization.

IL-12p40: A subunit of interleukin-12 (IL-12), a cytokine that acts on T cells and natural killer cells, and has a broad array of biological activities. IL-12 is a disulfide-linked heterodimer composed of the 40 kD cytokine receptor like subunit (IL-12p40) encoded by the IL12B gene, and a 35 kD subunit encoded by IL12A. IL-12p40 is expressed by activated macrophages that serve as an essential inducer of Th1 cell development. This cytokine has been found to be important for sustaining a sufficient number of memory/effector Th1 cells to mediate long-term protection to an intracellular pathogen. IL-12p40 is also known as interleukin 12B (natural killer cell stimulatory factor 2, cytotoxic lymphocyte maturation factor 2, p40), IL12B, CLMF, NKSF, CLMF2 and NKSF2. See NCBI Gene ID 3593 for human IL-12p40.

Intrathecal administration: Administration into the subarachnoid space under the arachnoid membrane of the brain or spinal cord through which the cerebral spinal fluid flows. For example, intrathecal delivery can be accomplished by delivery through a needle into the subarachnoid space of the spinal cord or brain (such as by lumbar puncture), or intraventricularly into the cerebrospinal fluid (CSF) in one of the ventricles of the brain for subsequent flow through the subarachnoid space of the brain or spinal cord.

Intravenous administration: Administration into a vein.

Measuring the level: As used herein, measuring the level of particular protein (such as IL-12p40 or CXCL13) refers to quantifying the amount of the protein present in a sample (such as a CSF sample). Quantification can be either numerical or relative. Detecting expression of the protein can be achieved using any method known in the art or described herein, such as by ELISA.

Meningeal inflammation: Inflammation of the meninges, the membranes that cover the brain and spinal cord.

Multiple sclerosis: An autoimmune disease classically described as a central nervous system white matter disorder disseminated in time and space that presents as relapsing-remitting illness in 80-85% of patients. Diagnosis can be made by brain and spinal cord magnetic resonance imaging (MRI), analysis of somatosensory evoked potentials, and analysis of cerebrospinal fluid to detect increased amounts of immunoglobulin or oligoclonal bands. MRI is a particularly sensitive diagnostic tool. MRI abnormalities indicating the presence or progression of MS include hyperintense white matter signals on T2-weighted and fluid attenuated inversion recovery images, gadolinium enhancement of active lesions, hypointensive “black holes” (representing gliosis and axonal pathology), and brain atrophy on T1-weighted studies. Serial MRI studies can be used to indicate disease progression. Relapsing-remitting multiple sclerosis (RRMS) is a clinical course of MS that is characterized by clearly defined, acute attacks with full or partial recovery and no disease progression between attacks. Secondary-progressive multiple sclerosis (SPMS) is a clinical course of MS that initially is relap sing-remitting, and then becomes progressive at a variable rate, possibly with an occasional relapse and minor remission. Primary progressive multiple sclerosis (PPMS) presents initially in the progressive form.

Pharmaceutical agent or drug: A chemical compound or composition capable of inducing a desired therapeutic or prophylactic effect when properly administered to a subject.

Pharmaceutically acceptable carriers: The pharmaceutically acceptable carriers useful in the methods disclosed herein are conventional. Remington's Pharmaceutical Sciences, by E. W. Martin, Mack Publishing Co., Easton, Pa., 15th Edition (1975), describes compositions and formulations suitable for pharmaceutical delivery of antibodies, such as rituximab.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain non-toxic auxiliary substances, such as wetting or emulsifying agents, preservatives, salts, amino acids, and pH buffering agents and the like, for example sodium or potassium chloride or phosphate, Tween, sodium acetate or sorbitan monolaurate.

Rituximab: A chimeric monoclonal antibody that specifically binds CD20, which is primarily found on the surface of B cells. Rituximab is used in the treatment of lymphoma, leukemia, transplant rejection and autoimmune disorders, including multiple sclerosis. Rituximab is sold under the trade names RITUXAN™ and MABTHERA™. Rituximab is a genetically engineered monoclonal antibody with murine light and heavy chain variable regions, and human gamma 1 heavy chain and kappa light chain constant regions. The chimeric antibody is composed of two heavy chains of 451 amino acids and two light chains of 213 amino acids and has an approximate molecular weight of 145 kD. Rituximab was genetically engineered using the murine 2B8 antibody and is described in, for example, U.S. Pat. No. 6,455,043; U.S. Pat. No. 5,736,137; U.S. Pat. No. 5,843,439; and U.S. Pat. No. 5,776,456, each of which is herein incorporated by reference. The 2B8 hybridoma is deposited with the ATCC under deposit number HB-11388.

Sample or biological sample: As used herein, a “sample” obtained from a subject refers to a cell, fluid or tissue sample. Bodily fluids include, but are not limited to, cerebral spinal fluid, blood, serum, urine and saliva.

Subject: A human or non-human animal. In one embodiment, the subject has multiple sclerosis.

Symptom and sign: Any subjective evidence of disease or of a subject's condition, i.e., such evidence as perceived by the subject; a noticeable change in a subject's condition indicative of some bodily or mental state. A “sign” is any abnormality indicative of disease, discoverable on examination or assessment of a subject. A sign is generally an objective indication of disease. Signs include, but are not limited to any measurable parameters such as tests for immunological status or the presence of lesions in a subject with multiple sclerosis.

Therapeutically Effective Amount: A dose sufficient to prevent advancement, or to cause regression of the disease, or which is capable of reducing symptoms caused by the disease, such as multiple sclerosis.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. “Comprising A or B” means including A, or B, or A and B. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### III. Intrathecal Administration of B Cell-Depleting Agents for the Treatment of Multiple Sclerosis

There are currently no therapies that are effective for patients with secondary progressive multiple sclerosis (SPMS) or primary progressive multiple sclerosis (PPMS) who do not have evidence for focal brain inflammation measured by contrast-enhancing lesion (CEL), as determined by brain MRI. Rituximab, an anti-CD20 monoclonal antibody that depletes B cells effectively, decreases CEL in relapsing-remitting MS (RRMS), but does not affect progression of disability in progressive MS because it does not deplete B cells in the intrathecal compartment in these patients. Therefore, the present disclosure provides for the use of an intrathecally (IT) introduced B cell depleting agent, such as an anti-CD20 monoclonal antibody, for example rituximab or ocrelizumab, that depletes B cells in the intrathecal compartment, leading to inhibition of T cell activation within the intrathecal compartment. Other B cell depleting agents include, but are not limited to, anti-CD19 monoclonal antibodies and anti-CD22 monoclonal antibodies, for example epratuzumab.

Provided herein is a method of treating MS by intrathecally administering a B cell depleting agent, such as a monoclonal antibody specific for a B cell surface antigen. In some embodiments, the method includes selecting a subject with MS and administering to the subject a therapeutically effective amount of the B cell depleting agent intrathecally, thereby treating the subject with MS. In some examples, the B cell surface antigen is CD20. In other examples, the B cell surface antigen is CD19 or CD22. In some examples, the method further includes administering the B cell depleting agent intravenously. In some examples, the method includes intrathecal administration of more than one B cell depleting agent, such as two or three B cell depleting agents.

In some embodiments, provided herein is a method of treating a subject with MS by selecting a subject with MS and administering to the subject a therapeutically effective amount of the B cell depleting agent, for example an anti-CD20 monoclonal antibody (such as rituximab or ocrelizumab), an anti-CD19 monoclonal antibody or anti-CD22 monoclonal antibody (such as epratuzumab), into the CSF which circulates through the subarachnoid space, thereby treating the subject with MS. For example, the monoclonal antibody is delivered intrathecally (for example via lumbar puncture) for direct delivery to the subarachnoid space. Alternatively, the antibody is delivered into one of the ventricles of the brain (for example, through an infusion pump) so that the antibody circulates in the CSF to the subarachnoid space of the brain and spinal cord. In an alternative embodiment, the antibody is administered intranasally.

Selecting a subject with MS can include any standard diagnostic method, such as, but not limited to, brain and spinal cord magnetic resonance imaging (MRI), analysis of somatosensory evoked potentials, or analysis of cerebrospinal fluid to detect increased amounts of immunoglobulin or oligoclonal bands.

In some embodiments, the antibody is rituximab and the therapeutically effective amount of rituximab administered intrathecally is about 10 mg to about 50 mg per dose, or about 20 mg to about 30 mg per dose, or about 25 mg per dose. In particular examples, the therapeutically effective amount of rituximab administered intrathecally is about 10 mg, about 15 mg, about 20 mg, about 25 mg, about 30 mg, about 35 mg, about 40 mg, about 45 mg or about 50 mg.

In some embodiments, the subject is administered a single dose of the B cell depleting agent, such as rituximab, directly into the CSF, for example intrathecally. In other embodiments, the subject is administered multiple doses of the B cell depleting agent, such as rituximab, into the CSF, for example intrathecally, such as 2, 3, 4, 5, 6, 7, 8, 9 or 10 doses. In particular examples, the subject is administered two or three doses of rituximab into the CSF, for example, the subject is administered two or three doses of about 25 mg rituximab into the CSF intrathecally.

The timing of administration of the multiple doses can be determined by a medical practitioner (such as by evaluating the progression of the disease and/or evaluating B cell depletion and T cell activation in the intrathecal compartment). In some embodiments in which two intrathecal doses are administered, the intrathecal doses are administered about 8 to about 16 months apart, such as about 10 to about 14 months apart, such as about 12 months apart.

In some embodiments in which three intrathecal doses are administered, the first and second intrathecal doses are administered about 1 to about 2 months apart, such as about six weeks apart; and/or the first and third intrathecal doses are administered about 8 to about 16 months apart, such as about 10 to about 14 months apart, such as about 12 months apart.

In one example, the subject is administered intrathecal rituximab (or any B cell depleting agent) only once a year for multiple years (for example, at least 2, 3, 4, 5 or more years) as needed to limit the progression of MS.

In some embodiments, the subject is further administered a therapeutically effective amount of a B cell depleting agent intravenously. In some examples, the same B cell depleting agent that is administered intrathecally is administered intravenously. In other examples, the B cell depleting agent administered intravenously is different than the B cell depleting agent administered intrathecally.

In some examples, the B cell depleting agent is rituximab and the therapeutically effective amount of rituximab administered intravenously is about 150 mg to about 300 mg per dose, about 200 mg to about 250 mg per dose or about 200 mg per dose. In other examples, the intravenous dose of rituximab is about 200 mg, about 300 mg, about 400 mg, about 500 mg, about 600 mg, about 700 mg, about 800 mg, about 900 mg or about 1000 mg.

In some examples, the first intrathecal dose is administered simultaneously with a first intravenous dose of the B cell depleting agent. In other examples, the first intrathecal dose is administered prior to the first intravenous dose, such as about 2-24 hours prior, for example about 2, about 4 or about 6 hours prior.

In particular examples, the subject is administered a single dose of a B cell depleting agent intravenously. In other examples, the subject is administered multiple doses of the B cell depleting agent intravenously, such as two doses, three doses or four doses. In one non-limiting example, the subject is administered two doses of about 200 mg rituximab intravenously.

In some embodiments, the two intravenous doses are administered about one week, about two weeks, about three weeks, about four weeks, about five weeks or about six weeks apart. In some examples, the two intravenous doses are administered about two weeks parts. In other examples, the two intravenous doses are administered about one month apart.

In some embodiments, the subject has secondary progressive MS. In other embodiments, the subject has primary progressive MS or relapsing remitting MS.

In one embodiment, provided is a method of treating a subject with MS by selecting a subject with secondary progressive MS (SPMS); administering to the subject a first dose of 25 mg rituximab into the CSF and a first intravenous dose of 200 mg rituximab, wherein the first dose of rituximab into the CSF and the first intravenous dose of rituximab are administered less than 24 hours apart (such as simultaneously); administering to the subject a second intravenous dose of 200 mg rituximab about two weeks following the first intravenous dose; and administering to the subject a second dose of 25 mg rituximab into the CSF about 12 months following the first dose into the CSF, thereby treating the subject with MS.

In one example, provided herein is a method of treating a subject with MS, comprising: selecting a subject with SPMS; administering to the subject a first intrathecal dose of 25 mg rituximab simultaneously with a first intravenous dose of 200 mg rituximab; administering to the subject a second intravenous dose of 200 mg rituximab about two weeks following the first intravenous dose; and administering to the subject a second intrathecal dose of 25 mg rituximab about 12 months following the first intrathecal dose, thereby treating the subject with MS.

In some embodiments of the disclosed methods, a first intrathecal dose and a first intravenous dose are administered simultaneously, on the same day, one day apart, two days apart or three days apart. In some embodiments, rituximab is administered intrathecally first, followed by intravenous administration. When administered is on the same day, intravenous administration can follow intrathecal administration by about 30 minutes, about 60 minutes, about 2 hours, about 4 hours, about 6 hours, about 8 hours, about 12 hours or about 24 hours.

In one embodiment, provided is a method of treating a subject with MS by selecting a subject with SPMS; administering to the subject a first dose of 25 mg rituximab into the CSF and a first intravenous dose of 200 mg rituximab, wherein the first dose of rituximab into the CSF and the first intravenous dose of rituximab are administered about 2-24 hours apart; administering to the subject a second intravenous dose of 200 mg rituximab about two weeks following the first intravenous dose; administering to the subject a second dose of 25 mg rituximab into the CSF about six weeks following the first dose into the CSF; and administering to the subject a third dose of 25 mg rituximab into the CSF about 12 months following the first dose into the CSF, thereby treating the subject with MS. In some examples, the first dose of rituximab into the CSF is administered about 2, about 4, about 6 or about 8 hours prior to the first intravenous dose of rituximab. In one non-limiting example, the first dose of rituximab into the CSF is administered about 4 hours prior to the first intravenous dose of rituximab.

As described above, selecting a subject with MS can include any standard diagnostic method, such as, but not limited to, brain and spinal cord MRI, analysis of somatosensory evoked potentials, or analysis of cerebrospinal fluid to detect increased amounts of immunoglobulin or oligoclonal bands. SPMS is particularly characterized by an initial relapsing-remitting disease, but then becomes progressive at a variable rate.

In some embodiments of the treatment methods, selecting the subject with MS comprises selecting a subject in whom the level of IL-12p40, CXCL13 or both in the CSF of the subject is increased relative to a control. In other embodiments, selecting the subject with MS comprises measuring the level of IL-12p40, CXCL13 or both in the CSF of the subject, as disclosed herein. In some examples, the control is a sample from a healthy subject. In other examples, the control is a reference standard.

In yet other embodiments, selecting the subject with MS comprises performing one or more of brain and spinal cord MRI(s), analysis of somatosensory evoked potentials, analysis of cerebrospinal fluid to detect increased amounts of immunoglobulin or oligoclonal bands, or any other art-accepted method of diagnosing a subject with MS.

### IV. Use of IL-12p40 and CXCL12 in CSF as Biomarkers to Stratify patients for Treatment with IT Immunomodulators

Disclosed herein is the finding that increased levels of IL-12p40 and/or CXCL13 protein in the CSF of MS patients can be used to identify progressive MS patients with meningeal inflammation. In particular, CSF levels of IL-12p40 and CXCL13 can be used for stratification of patients with progressive multiple sclerosis and closed blood brain barrier (BBB) for treatment with intrathecally (IT) administered immunomodulators, such as IT rituximab (or IT administration of another B cell depleting agent).

A subgroup of patients with progressive MS has prominent meningeal inflammation (including tertiary lymphoid follicles) that is likely driving development of clinical disability. There are currently no means for identifying these patients. It is disclosed herein that increased levels of IL-12p40 and CXCL13, detectable in approximately 23% of patients with progressive MS, are biomarkers of meningeal inflammation. Thus, these biomarkers can be utilized to stratify these patients for the treatment with IT B cell depleting agents, such as rituximab. These biomarkers can also be utilized for following treatment efficacy.

Thus, provided herein is a method of selecting a patient with progressive MS as a candidate for treatment with an intrathecal immunomodulatory therapy, comprising measuring the level of IL-12p40, CXCL13, or both in the CSF of the patient. An increase in the level of IL-12p40, CXCL13, or both relative to a control, indicates the subject is a candidate for treatment with an intrathecal immunomodulatory therapy.

Also provided is a method of identifying a progressive MS patient as having meningeal inflammation by measuring the level of IL-12p40, CXCL13, or both in the CSF of the patient. An increase in the level of IL-12p40, CXCL13, or both relative to a control, indicates that the MS patient has meningeal inflammation.

In some embodiments, the increase in IL-12p40, CXCL13, or both is about 1.5-fold, about 2-fold, about 3-fold or about 4-fold relative to the control.

In some embodiments, the disclosed methods further include administering to the patient an intrathecal immunomodulatory therapy. In some examples, the intrathecal immunomodulatory therapy comprises intrathecal administration of a B cell depleting agents, such as rituximab.

Further provided is a method of evaluating the effectiveness of a therapy for treating progressive MS by measuring the level of IL-12p40, CXCL13, or both in the CSF of the patient before and after treatment. A decrease in the level of IL-12p40, CXCL13, or both after treatment indicates the therapy is effective for treating progressive MS.

In some embodiments, the progressive MS is primary progressive MS. In other embodiments, the progressive MS is secondary progressive MS.

In some embodiments, the level of IL-12p40, CXCL13, or both, is measured by immunoassay, such as by ELISA, Western blot, cytometric bead assay or radioimmunoprecipitation assay.

A. Methods for Detection of IL-12p40 and CXCL13 Protein

Antibodies specific for IL-12p40 or CXCL13 can be used for detection and quantification of IL-12p40 or CXCL13 by one of a number of immunoassay methods that are well known in the art, such as those presented in Harlow and Lane (Antibodies, A Laboratory Manual, CSHL, New York, 1988). Methods of constructing such antibodies are known in the art.

Any standard immunoassay format (such as ELISA, Western blot, cytometric bead assay or RIA) or newer quantifiable proteomic approaches based on mass spectrometry can be used to measure protein levels. Thus, IL-12p40 or CXCL13 protein levels in a sample (such as a CSF sample) can readily be evaluated using these methods. General guidance regarding such techniques can be found in Bancroft and Stevens (Theory and Practice of Histological Techniques, Churchill Livingstone, 1982) and Ausubel et al. (Current Protocols in Molecular Biology, John Wiley & Sons, New York, 1998).

For the purposes of quantifying IL-12p40 or CXCL13, a biological sample of the subject, such as CSF, can be used. Quantification of IL-12p40 or CXCL13 protein can be achieved by immunoassay methods known in the art. The amount IL-12p40 or CXCL13 protein can be assessed in samples from MS patients and/or in samples from healthy subjects. The amounts of IL-12p40 or CXCL13 protein in the sample can be compared to a control, such as the levels of the proteins in CSF from a healthy subject or other control (such as a standard value or reference value). A significant increase or decrease in the amount can be evaluated using statistical methods known in the art.

B. IL-12p40 or CXCL13-Specific Antibodies

For immunoassay methods, commercially available ELISA kits for detection of IL-12p40 or CXCL13, or commercially available antibodies to IL-12p40 or CXCL13 can be utilized. Moreover, methods of making polyclonal and monoclonal antibodies are well known in the art. Polyclonal antibodies, antibodies which consist essentially of pooled monoclonal antibodies with different epitopic specificities, as well as distinct monoclonal antibody preparations are included. The preparation of polyclonal antibodies is well known to those skilled in the art (see, for example, Green et al., “Production of Polyclonal Antisera,” in: Immunochemical Protocols, pages 1-5, Manson, ed., Humana Press, 1992; Coligan et al., “Production of Polyclonal Antisera in Rabbits, Rats, Mice and Hamsters,” in: Current Protocols in Immunology, section 2.4.1, 1992).

The preparation of monoclonal antibodies likewise is conventional (see, for example, Kohler & Milstein, Nature 256:495, 1975; Coligan et al., sections 2.5.1-2.6.7; and Harlow et al. in: Antibodies: a Laboratory Manual, page 726, Cold Spring Harbor Pub., 1988). Briefly, monoclonal antibodies can be obtained by injecting mice with a composition comprising an antigen, verifying the presence of antibody production by removing a serum sample, removing the spleen to obtain B lymphocytes, fusing the B lymphocytes with myeloma cells to produce hybridomas, cloning the hybridomas, selecting positive clones that produce antibodies to the antigen, and isolating the antibodies from the hybridoma cultures. Monoclonal antibodies can be isolated and purified from hybridoma cultures by a variety of well-established techniques. Such isolation techniques include affinity chromatography with Protein-A Sepharose, size-exclusion chromatography, and ion-exchange chromatography (see, e.g., Coligan et al., sections 2.7.1-2.7.12 and sections 2.9.1-2.9.3; Barnes et al., Purification of Immunoglobulin G (IgG), in: Methods in Molecular Biology, Vol. 10, pages 79-104, Humana Press, 1992).

Antibodies include intact molecules as well as fragments thereof, such as Fab, F(ab′)₂, and Fv which are capable of binding the epitopic determinant. These antibody fragments retain some ability to selectively bind with their antigen or receptor and are defined as follows:

(1) Fab, the fragment which contains a monovalent antigen-binding fragment of an antibody molecule, can be produced by digestion of whole antibody with the enzyme papain to yield an intact light chain and a portion of one heavy chain;

(2) Fab′, the fragment of an antibody molecule can be obtained by treating whole antibody with pepsin, followed by reduction, to yield an intact light chain and a portion of the heavy chain; two Fab′ fragments are obtained per antibody molecule;

(3) (Fab′)₂, the fragment of the antibody that can be obtained by treating whole antibody with the enzyme pepsin without subsequent reduction; F(ab′)₂ is a dimer of two Fab′ fragments held together by two disulfide bonds;

(4) Fv, defined as a genetically engineered fragment containing the variable region of the light chain and the variable region of the heavy chain expressed as two chains; and

(5) Single chain antibody, defined as a genetically engineered molecule containing the variable region of the light chain, the variable region of the heavy chain, linked by a suitable polypeptide linker as a genetically fused single chain molecule.

Methods of making these fragments are known in the art (see for example, Harlow and Lane, Antibodies: A Laboratory Manual, Cold Spring Harbor Laboratory, New York, 1988). For example, antibody fragments can be prepared by proteolytic hydrolysis of the antibody or by expression in E. coli of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly (see U.S. Pat. No. 4,036,945 and U.S. Pat. No. 4,331,647, and references contained therein; Nisonhoff et al., Arch Biochem Biophys 89:230, 1960; Porter, Biochem J 73:119, 1959; Edelman et al., Methods in Enzymology, Vol. 1, page 422, Academic Press, 1967; and Coligan et al. at sections 2.8.1-2.8.10 and 2.10.1-2.10.4).

The following examples are provided to illustrate certain particular features and/or embodiments. These examples should not be construed to limit the disclosure to the particular features or embodiments described.

### EXAMPLES

### Example 1

### Use of Intrathecal (IT) Rituximab for the Treatment of Secondary-Progressive Multiple Sclerosis (SPMS)

This example describes a human clinical trial for the treatment of SPMS using combination intravenous (IV)/intrathecal (IT) administration of rituximab. Data obtained during this trial demonstrate that low dose IV rituximab in combination with 25 mg IT rituximab significantly inhibits intrathecal activation of the adaptive immune response in patients with SPMS.

### BACKGROUND

Secondary-progressive multiple sclerosis (SPMS) is the chronic phase of multiple sclerosis (MS). The majority of people who have relapsing-remitting MS eventually develop SPMS. There are currently no effective treatments for SPMS. Researchers are interested in determining whether the drug rituximab, which is used to treat rheumatoid arthritis and some types of cancer, is able to target certain white blood cells that are thought to play a role in the progression of SPMS. To ensure that the rituximab will reach the brain and spinal cord, participants will receive it by intravenous drip and by intrathecal injection (through a lumbar puncture into the cerebrospinal fluid).

### Objectives:

To evaluate the safety and effectiveness of combined intravenous and intrathecal rituximab in individuals with secondary-progressive multiple sclerosis.

### Eligibility:

Individuals between 18 and 65 years of age who have been diagnosed with SPMS and have been off any form of immunosuppressive therapy for at least 3 months are eligible.

### Design:

The study involves a 1-year pretreatment baseline series of visits, followed by a 2-year treatment period. Participants provide blood samples throughout treatment as directed by the study researchers, and additional studies may be performed during the study period if participants consent to further investigation.

Baseline Visits:
Visit 1: Participants provide blood samples and have a magnetic resonance imaging (MRI) scan of the brain.
Visits 2 and 3: In addition to providing blood samples, participants have an MRI scan of the spine, additional tests of vision and motor skills, and a lumbar puncture to collect a sample of cerebrospinal fluid. Participants are randomly assigned to receive either rituximab or a placebo.
Visit 4: In addition to providing blood samples, participants have an MRI scan of the brain and a skin biopsy.

Treatment Visits:
Visit 5: Participants are admitted for a 2-day inpatient stay, and have MRI scans, vision and motor skills tests, and blood samples on the first day. On the second day, participants receive rituximab or placebo by both intravenous drip and through a lumbar puncture, and are discharged on the following day after overnight monitoring.
Visit 6: Two weeks after Visit 5, participants have an overnight stay to receive rituximab or placebo by intravenous drip only.
Visit 7: Six months after Visit 6, participants have MRI scans and provide blood samples.
Visit 8: One year after Visit 5, participants have another 2-day inpatient stay. On the first day, the same procedures performed described for Visit 5 are repeated; on the second day, participants receive rituximab or placebo through a lumbar puncture only, and are discharged on the following day after overnight monitoring.
Visit 9: Six months after Visit 8, participants have MRI scans and provide blood samples.
Visit 10: Six months after Visit 9, participants have MRI scans and provide blood samples.
After the end of the study, participants continue with standard care for SPMS.

### Study Type: Interventional

### Study Design Allocation: Randomized

Endpoint Classification: Safety/Efficacy Study
Intervention Model: Parallel Assignment
Masking: Double-Blind
Primary Purpose Treatment

### Primary Outcome Measures:

Individualized brain atrophy progression between the rituximab and placebo groups after 2 years of treatment is the default primary outcome measure. If predetermined analysis shows that one of the secondary outcome measures has higher z-score than brain atrophy measurement, the secondary outcome measure with highest sensitivity and specificity (as determined by analysis of first 30 patients during 1 year of pre-treatment baseline) is selected as new primary outcome.

### Secondary Outcome Measures:

Quantitative MRI markers
Clinical/paraclinical markers

### DETAILED DESCRIPTION

Objective: This study addresses the safety and efficacy of combined systemic and intrathecal (IT) B cell-depleting therapy (i.e. anti-CD20, rituximab) in patients with secondary-progressive multiple sclerosis (SPMS). The longitudinal data identify the most sensitive outcome measures and trial design for future Phase II trials for SPMS patients and elucidate the mechanism of action of rituximab on the human immune system.

Study Population Patients with SPMS and mild to moderate level of clinical disability, who have no medical contraindication to IT or intravenous (IV) administration of rituximab.

Design: This is double blind, placebo-controlled, single center, baseline versus treatment, Phase I/II clinical trial of IV and IT rituximab in SPMS patients.

Outcome Measures Quantitative neuroimaging measures of central nervous system (CNS: 1.e. brain and spinal cord) tissue destruction and clinical and functional (i.e. electrophysiological) measures of neurological disability are collected every 6-12 months. Additionally, biomarkers focusing on analysis of cerebral spinal fluid (CSF) B cells and immunological responses to EBV are collected at baseline and during treatment. The trial is currently powered using progression of brain atrophy as detected by SIENA methodology as the primary outcome measure. However, the trial has an adaptive design: it incorporates analysis of the progression of CNS tissue destruction, as measured by quantitative MRI markers, and clinical/paraclinical markers, defined as secondary outcome measures, in the first 30 enrolled patients during the one year pre-treatment baseline prior to randomization. All defined outcome measures collected in the first 30 enrolled patients will be transformed into z-scores and compared for the robustness of longitudinal change over the coefficient of variation. As a result, the primary outcome measure of this trial will be the comparison of individualized rates of brain atrophy progression between the rituximab and placebo groups after 2 years of treatment, unless the predetermined analysis establishes that one of the secondary outcome measures has a higher z-score than the brain atrophy measurement. In this case, the primary outcome would be the efficacy of rituximab versus placebo in inhibiting patient-specific slopes of functional or structural deterioration as measured by this more sensitive biomarker of CNS tissue destruction.

### Eligibility

### Ages Eligible for Study: 18 Years to 65 Years

### Genders Eligible for Study: Both

### Accepts Healthy Volunteers: No

### Criteria

### INCLUSION CRITERIA:

MS as defined by the modified McDonald's criteria (Polman, Reingold et al. 2005)

SPMS as documented by notes of the referring neurologist: lack of MS relapse for the past 1 year and non-remitting/sustained (>3 months) progression of disability

Age 18-65, inclusive

EDSS 3.0 to 7.0, inclusive

Able to provide informed consent

Willing to participate in all aspects of trial design and follow-up

Lack of CEL on all MRIs performed within the last 12 months or if patient has CEL, then documentation that they tried and failed or could not tolerate FDA approved disease modifying therapies (DMTh)
Not receiving any DMTh (such as IFN-beta preparation, glatiramer acetate, corticosteroid, natalizumab, immunosuppressive agents or experimental therapeutics) for a period of at least 3 months before enrollment in the study -Agreeing to commit to the use of a reliable/accepted method of birth control (i.e. hormonal contraception (birth control pills, injected hormones, vaginal ring), intrauterine device, barrier methods with spermicide (diaphragm with spermicide, condom with spermicide) or surgical sterilization (hysterectomy, tubal ligation, or vasectomy in a partner)) during enrollment in the study and through 12 months after the last dose of study drug

### Exclusion Criteria:

RRMS or PPMS

Evidence of clearly documented MS relapse within the last 1 year

Alternative diagnoses that can explain neurological disability and MRI findings

Clinically significant medical disorders that, in the judgment of the investigators could cause CNS tissue damage, limit its repair, expose the patient to undue risk of harm or prevent the patient from completing the study (such as, but not limited to cerebrovascular disease, ischemic cardiomyopathy, clotting disorder, brittle diabetes, neurodegenerative disorder)

Pregnant or breastfeeding female

History or sign of congenital or acquired immunodeficiency or chronic infections, such as HIV/AIDS, Hepatitis A, B or C, HTLV-1 carrier and others that would expose patient to risks of pathogen reactivation associated with rituximab treatment

Abnormal screening/baseline blood tests exceeding any of the limits defined below:
1. Serum alanine transaminase or aspartate transaminase levels which are greater than three times the upper limit of normal values.
2. Total white blood cell count<3 000/mm³
3. Platelet count<85 000/mm³
4. Serum creatinine level>2.0 mg/dl and eGFR (glomerular filtration rate)<60
5. Serological evidence of HIV, HTLV-1 or active hepatitis A, B or C
6. Positive pregnancy test
7. Positive CSF or serum quantitative PCR for JC virus on CSF collected from the baseline spinal tap (test will be performed by CLIA certified laboratory of Gene Major, NINDS)
8. Total serum IgG<600 mg/dl (nl 642-1730 mg/dl) or total serum IgM<30 mg/dl (nl 34-342 mg/dl) as these Ig deficiencies would suggest underlying abnormalities with B cell function/maturation

### Results

The effect of IT and IV rituximab treatment on B cell and T cell responses in MS patients was evaluated 3 months after treatment. Patients were administered a single dose (25 mg) of IT rituximab and a first IV dose (200 mg). After two weeks, patients received a second dose (200 mg) of IV rituximab. B and T cell responses were evaluated before, immediately after and 3 months after treatment. Four patients have been administered the above described treatment of IT and IV rituximab. Rituximab was well tolerated in all patients. For three patients, 3-month follow-up data is available.

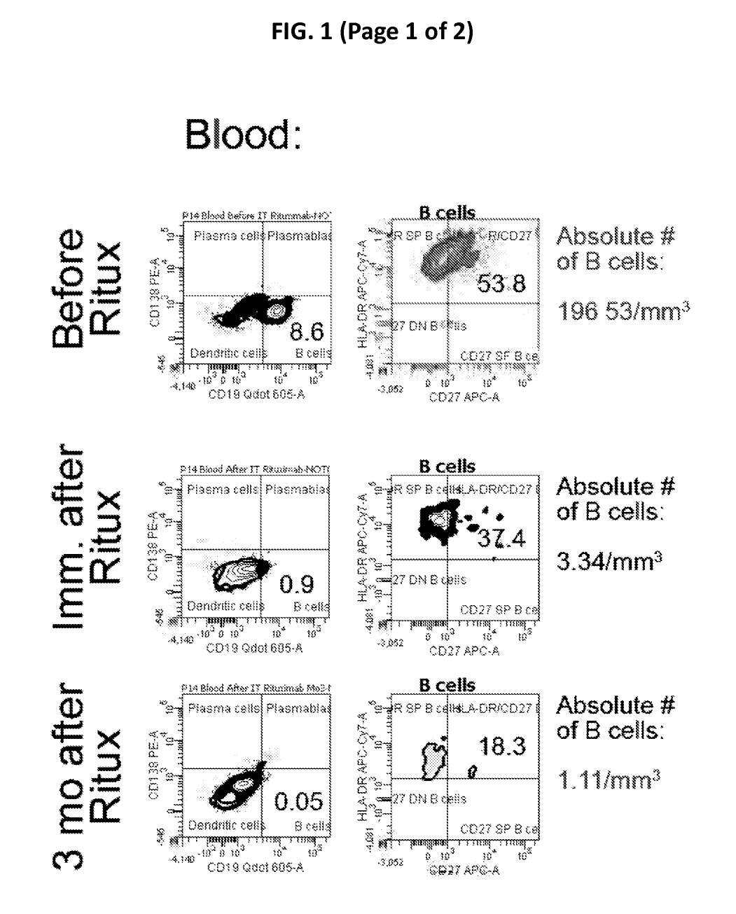

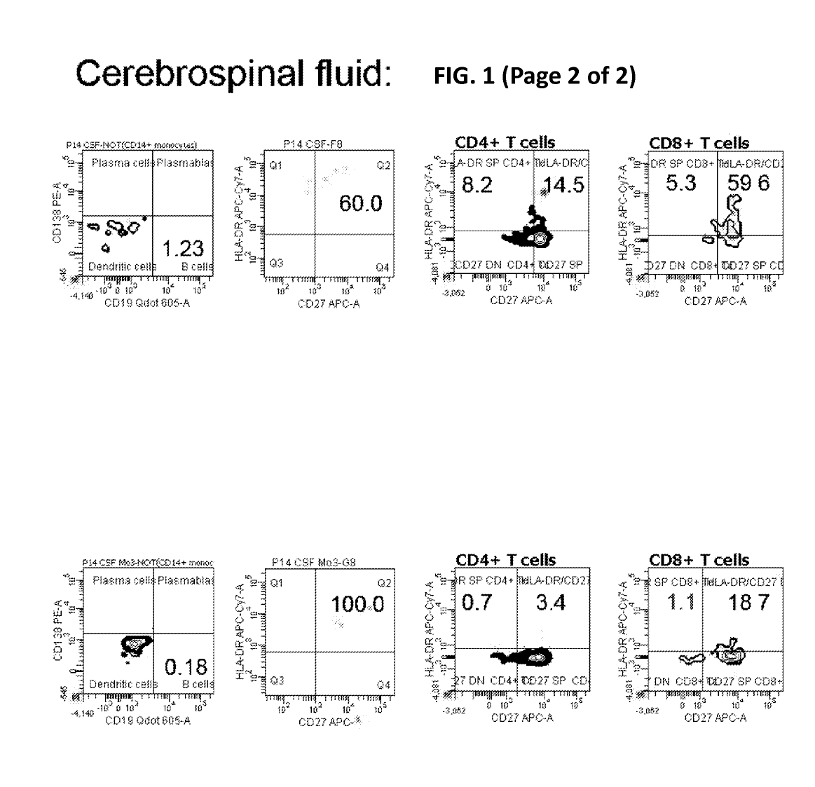

As shown in FIG. 1, in the first patient, IV rituximab treatment led to 99.5% depletion of B cells from the systemic circulation (page 1), which is comparable to results obtained using a 5-fold higher dose of IV rituximab in a clinical study of RRMS patients. The reconstitution of the systemic compartment occurred mainly by naïve (CD27-) B cells. The same result was obtained in two additional patients (i.e. 99.5% depletion of B cells from the blood).

In the first patient, the selected IT dose (25 mg) led to 85% depletion of B cells from cerebrospinal fluid 3 months after administration (FIG. 1, page 2). The proportion of T cells with an in vivo activated phenotype (i.e. expressing HLA-DR) decreased 3 months after administration of IT rituximab by 82% for CD4+ T cells and by 70% for CD8+ T cells. Thus, IT rituximab significantly inhibits intrathecal activation of adaptive immune responses in patients with SPMS who have closed blood brain barrier, as evidenced by the lack of CEL on repeated brain MRI images.

In two additional patients, although 99.5% depletion of B cells in the blood was achieved, no significant depletion of B cells was observed in the CSF.

### CXCL13 Levels in the CSF

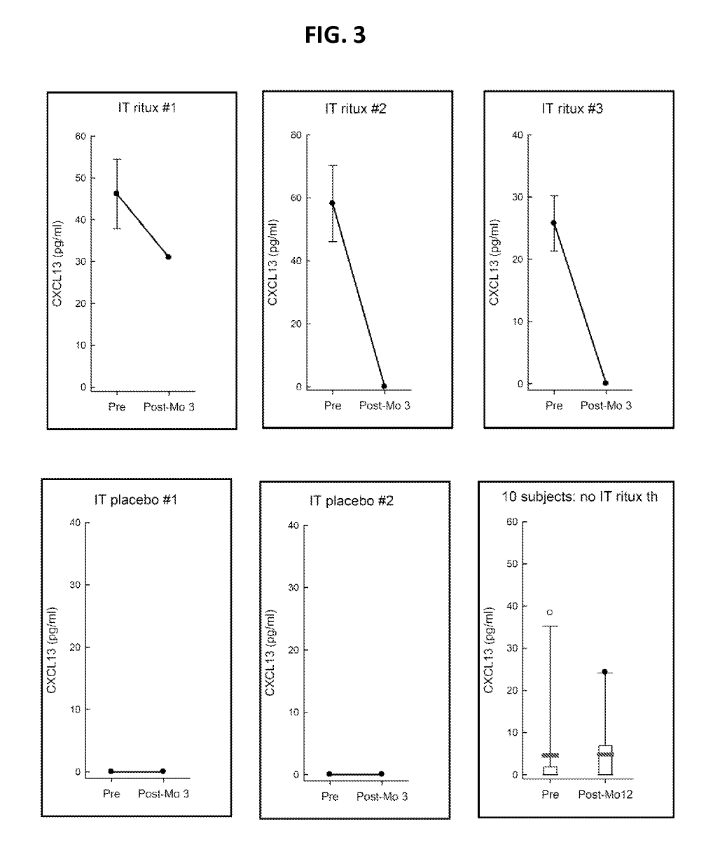

The level of CXCL13 in the CSF pre-treatment and 3 months post-treatment was evaluated in study participants. Three patients receiving IT rituximab and two patients treated with placebo were evaluated. In all three IT rituximab treated patients, the level of CXCL13 was decreased significantly 3 months post-treatment (FIG. 3 top). This result suggests that IT rituximab treatment is leading to depletion of B cells in the CNS.

In the two placebo-treated subjects, CXCL13 was not detectable pre-treatment or post-treatment (FIG. 3 bottom left and center). However, levels of CXCL13 were also evaluated in a cohort of ten progressive MS patients that are on a different (non-rituximab) MS therapy, which does not alter the immune system (FIG. 3, bottom right). In this cohort of 10 subjects, no significant decrease in CXCL13 level was observed 2 years after the first treatment.

### Example 2

### Modified IT rituximab clinical trial study design

This example describes a modified IT rituximab clinical trial design to improve depletion of B cells from the CSF.

The protocol described in Example 1 was amended to implement the following changes:
1. Institute delay between the first IT injection and first IV injection (at month (Mo) 0) of at least 4 hours; put patient into Trendelenburg position during those 4 hours to facilitate distribution of the drug over the brain convexities, where the meningeal B cell follicles are located. It is believed that the IV dose of rituximab creates high cytokine and chemokine burst, which then recruits immune effectors (i.e. NK cells and macrophages) that will mediate antibody-dependent cellular cytotoxicity (ADCC—which induces killing of B cells that have bound rituximab on cell surface) out of the intrathecal compartment and into the blood. The delay will allow at least 4 hours of killing of the CSF B cells, thereby increasing their depletion.
2. Add another dose of IT rituximab (25 mg) at Mo 1.5 (i.e. 6 weeks after first IT injection and 4 weeks after second IV injection). At this point the peripheral B cells are already depleted and no IV rituximab is being administered, so no cytokine/chemokine burst will be generated. Therefore, it is expected that the second dose will significantly enhance depletion of the B cells from the intrathecal compartment.
3. Lumbar puncture (LP) will be retained at 3 months to assess efficacy of B cell depletion from the CSF 6 weeks after the second IT dose.
   The modified clinical trial protocol is summarized below:

### Design

The study involves a 1-year pretreatment baseline series of visits, followed by a 2-year treatment period. Participants provide blood samples throughout treatment as directed by the study researchers, and additional studies may be performed during the study period if participants consent to further investigation.

### Baseline Visits

Visit 1: Participants provide blood samples and have a magnetic resonance imaging (MRI) scan of the brain.

Visits 2 and 3: In addition to providing blood samples, participants have an MRI scan of the spine, additional tests of vision and motor skills, and a lumbar puncture to collect a sample of cerebrospinal fluid. Participants will be randomly assigned to receive either rituximab or a placebo.

Visit 4: In addition to providing blood samples, participants have an MRI scan of the brain and a skin biopsy.

### Treatment Visits

Visit 5: Participants are admitted for a 2-day inpatient stay, and have MRI scans, vision and motor skills tests, and blood samples on the first day. On the second day, participants receive rituximab or placebo by both intravenous drip and through a lumbar puncture, and are discharged on the following day after overnight monitoring.

Visit 6: Two weeks after Visit 5, participants have an overnight stay to receive rituximab or placebo by intravenous drip only.

Visit 7: Approximately six months after Visit 6, participants have MRI scans and provide blood samples.

Visit 8: Approximately one year after Visit 5, participants have another 2-day inpatient stay. On the first day, the same procedures performed described for Visit 5 are repeated; on the second day, participants receive rituximab or placebo through a lumbar puncture only, and are discharged on the following day after overnight monitoring.

Visit 9: Approximately six months after Visit 8, participants have MRI scans and provide blood samples.

Visit 10: Approximately six months after Visit 9, participants have MRI scans and provide blood samples.

After the end of the study, participants will continue with standard care for SP-MS.

### Example 3

### Cerebrospinal Fluid IL-12p40 is a Biomarker of Intrathecal Inflammation in Multiple Sclerosis

Identification of CSF biomarkers could lead to a greater understanding of central nervous system (CNS) pathology in neuroimmunological disorders, including MS, and provide a means to determine the effectiveness of treatments. Many candidate CSF biomarkers have been proposed but the only one currently in clinical use is the quantification of intrathecal immunoglobulin synthesis, measured as CSF IgG index and oligoclonal bands (OCB). Since the most common form of MS, RRMS, is thought to be in large part an immune mediated disease, proteins secreted by cells of the immune system are particularly attractive candidate biomarkers. It has recently been reported that concomitantly with its robust inhibitory effect on CEL, daclizumab treatment also decreases CSF levels of IL-12p40 (Bielekova et al., Neurology 77(21):1877-1886, 2011). Therefore, it was investigated whether CSF levels of IL-12p40 could be utilized as a biomarker of intrathecal inflammation associated with MS. The utility of IL-12p40 was compared to CXCL13 and IL-8, which have been previously reported as biomarkers of intrathecal inflammation (Sellebjerg et al., Neurology 73:2003-2010, 2009; Krumbholz et al., Brain 129:200-211, 2006).

IL-12p40 is one subunit of the disulfide-linked heterodimer IL-12p70 (i.e. biologically active IL-12) and IL-23, produced mostly by cells of the myeloid lineage, such as monocytes, macrophages, microglia and myeloid dendritic cells. IL-12p40 mRNA has been found in autopsied brain lesions of MS patients (Windhagen et al., J Exp Med 182:1985-1996, 1995). CXCL13, a B cell chemoattractant is also produced by cells of myeloid lineage, such as follicular dendritic cells, monocytes and macrophages and is likewise expressed in MS lesions and in perivascular and meningeal infiltrates (Krumbholz et al., Brain 129:200-211, 2006). CXCL13 has been shown by independent groups to be increased in the CSF of patients with MS (Sellebjerg et al., Neurology 73:2003-2010, 2009; Krumbholz et al., Brain 129:200-211, 2006). In addition, there are effective therapies for RRMS that decrease CSF levels of CXCL13 or IL-8, a neutrophil chemoattractant (Mellergard et al., Mult Scler 16:208-217, 2010; Bartosik-Psujek and Stelmasiak, J Neural Transm 112:797-803, 2005). While some studies suggest that IL-8 is increased in the CSF of patients with MS (Mellergard et al., Mult Scler 16:208-217, 2010; Ishizu T et al., Brain 128:988-1002, 2005), others do not (Sørensen et al., J Clin Invest 103:807-815, 1999; Franciotta et al., J Neurol Sci 247:202-207, 2006; Saruhan-Direskeneli et al., J Neuroimmunol 145:127-134, 2003).

To evaluate whether or not IL-12p40 could serve as a CSF biomarker of MS disease activity, IL-12p40, CXCL13 and IL-8 were measured, in a blinded fashion, in two independent, prospectively-acquired cohorts of untreated patients and embedded controls. In the larger, confirmatory cohort, the relationship of these three CSF biomarkers to MS disease activity was assessed, measured as MRI CEL.

### Methods

### Patients

The CSF was collected from patients who were not receiving disease-modifying therapies (DMThs) and presented for diagnostic work-up of a putative CNS neuroimmunological disorder. Diagnosis of MS was made based on McDonald's criteria (McDonald et al., Ann Neurol 50:121-7, 2001). Alternative diagnoses were made based on clinical diagnostic tests and prospective follow-up. The demographic data and diagnostic categories for both cohorts (pilot cohort—WMS and confirmatory cohort—NIB) are depicted in Table 1.

Both cohorts were prospectively acquired under natural history protocols headed by the same investigator, but at two different institutions. CSF from both cohorts was processed using identical procedures: CSF samples were transported on ice and centrifuged (300×g for 10 minutes) within 15 minutes of collection. Cell-free supernatant was prospectively coded, aliquotted and cryopreserved at −80° C. until analysis. All analyses were performed blindly, and the diagnostic code was broken by the investigator after the collection of all data was completed.

### Protein Measurement

Commercially available ELISA kits were used to measure CXCL13 (DY801, R&D, USA) and IL-23 (BMS 2023/3, Bender MedSystems, Austria), while bead-based assays were used to measure IL-8 (558277, BD, USA) and IL-12p70 (558283, BD, USA). The detection limits for the CXCL13, IL-23, IL-8 and IL-12p70 assays were 62.5 pg/ml, 31.3 pg/ml, 19.5 pg/ml and 4.9 pg/ml, respectively. In the WMS cohort, IL-12p40 was measured with a bead-based assay (560154, BD, USA) that had a detection limit of 19.5 pg/ml. The IL-12p40 bead based assay was not deemed sensitive enough, so for the NIB cohort an ELISA (KHC0121, Invitrogen, USA) was used. The IL-12p40 ELISA uses an IL-12p70 standard and detects IL-12p40 and IL-12p70. Thus, after it was determined that no appreciable amount of IL-12p70 was present in the CSF, the standard curve was calculated based on the amount of IL-12p40 in each standard well. This calculation gave the IL-12p40 ELISA a sensitivity of 9.1 pg/ml. Only the linear part of the standard curve was used to derive results and assay variability. It was determined that the ranges of intra-assay coefficient of variances were 0-7.5%, 0-21.9% and 0-27.9% for CXCL13, IL-8 and IL-12p40, respectively. All values of IL-12p70 and IL-23 were below the detection limit of the assays, so intra-assay coefficients of variance were not calculated. The range of the inter-assay coefficient of variance for the IL-12p40 assays was 14.15-23.69%.

In order to measure some of the protein concentrations in the linear part of the standard curve, CSF supernatant sometimes had to be concentrated using Amicon Ultra 3 kDa filters (Millipore). Only IL-12p40 (40 kDa), CXCL13 (10 kDa) and IL-8 (6 kDa) were measured in concentrated CSF supernatant. In the WMS cohort, CSF supernatant was concentrated ten-fold and five-fold for IL-8 and CXCL13, respectively. In the NIB cohort, both chemokines were measured in CSF supernatant that was not concentrated. For measuring IL-12p40, CSF supernatant was concentrated ten-fold for the WMS cohort and four-fold for the NIB cohort. For IL-12p40 and IL-8 the medians (P=0.872 and P=0.266; Mann-Whitney rank sum) and the means (P=0.620 and P=0.356; t-test) of the IL-12p40 and IL-8 concentrations were the same for both cohorts, but for CXCL13, there was a statistically-significant increase in the NIB cohort in the median (62.5 versus 62.5, P<0.001) and mean (69.8 versus 146.8 P<0.001) CSF concentrations. Because of this, filtrates from the concentration step were evaluated; no detectable levels of any protein were observed. It was concluded that protein was not lost to concentration.

### Statistical Analyses

All statistical analyses were performed using SigmaPlot 10.0. Where concentrations of proteins were below the lower detection limit, the lower detection limit of the assay was used for calculating the statistics. A Mann-Whitney rank sum test was used to compare the median protein concentrations between two groups, and a Kruskal-Wallis One Way ANOVA on Ranks followed by a Dunn's post-test (DPT) was used to compare protein concentrations between more than two groups. To analyze potential linear relationships between protein concentrations and brain CEL, non-parametric Spearman correlations were used. The preset limit of statistical significance was p<0.05. Bonferroni correction was applied to adjust for multiple comparisons.

### Results

### Pilot (WMS) Cohort

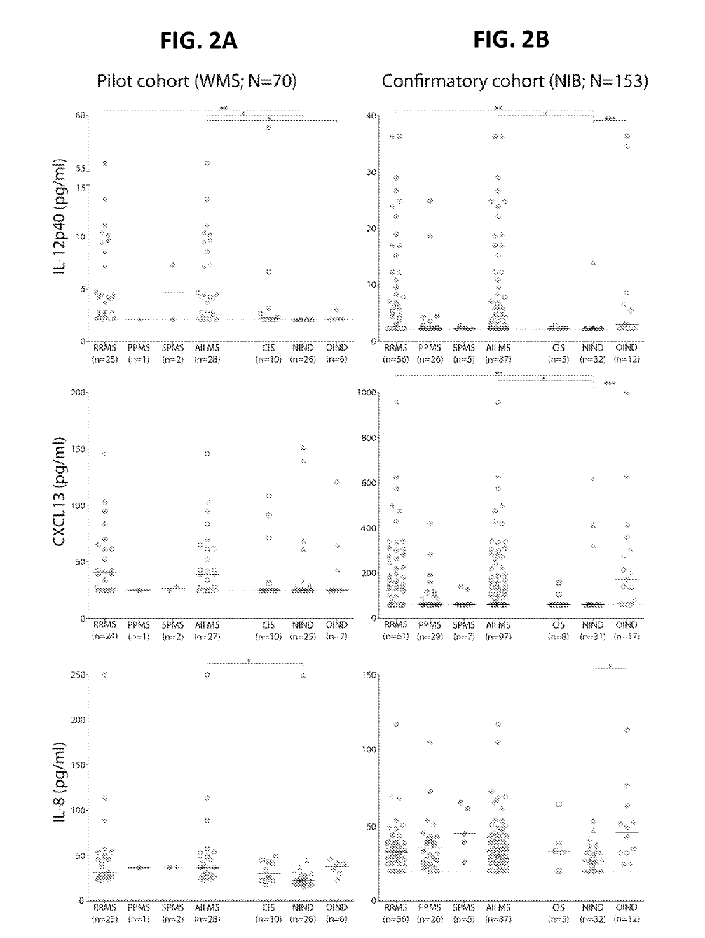

In the WMS cohort (N=70), MS patients (vast majority had RRMS) had a significantly higher concentration of CSF IL-12p40 than patients in the OIND (other inflammatory neurological diseases) or the NIND (non-inflammatory neurological diseases) group. No significant differences between groups were observed for CXCL13. MS patients had a significantly higher concentration of IL-8 than patients in the NIND group (FIG. 2A).

### Confirmatory (NIB) Cohort

To confirm these results and investigate a relationship with brain MRI measures of disease activity, protein concentrations were measured in the larger NIB cohort (N=167). In this cohort, MS patients had higher levels of IL-12p40 in comparison to NIND, but not OIND controls (FIG. 2B). In fact, the difference between the OIND and NIND groups reached statistical significance. Splitting the MS cohort into RRMS, PPMS and SPMS and considering six groups of patients, only the RRMS subgroup demonstrated statistically greater IL-12p40 than the NIND group (FIG. 2B).

IL-12p40 was not specific for MS, but using a cutoff of 2.4 pg/ml, IL-12p40 was quite specific for inflammatory diseases as it had a specificity of 0.97 (0.84-0.99 95% CI) and a positive likelihood ratio of 15.1 to discern between either MS, CIS or OIND and NIND. If the WMS cohort was included in the analysis, the specificity and positive likelihood ratio of IL-12p40 jumped to 0.98 (0.91-0.99 95% CI) and 29.9, respectively. However, in the NIB cohort, IL-12p40 had a sensitivity of only 0.47 (0.37-0.57 95% CI) and a negative likelihood ratio of 0.55.

IL-12p70 was measured in all patients in whom IL-12p40 was measured. No patients had detectable levels of IL-12p70, including three patients with greater than 30 pg/ml of IL-12p40 in their CSF. The detection limit for IL-12p70 (4.9 pg/ml) was comparable to that for IL-12p40 on four-fold concentrated CSF supernatant (2.1 pg/ml) leading to the conclusion that the IL-12p40 that was detected in the CSF of these patients was not a part of the heterodimer IL-12p70. IL-23 levels were measured in 36 NIB patients whose CSF had detectable levels of IL-12p40, and after none of these samples had detectable levels of IL-23, including those from the three patients with greater than 30 pg/ml of IL-12p40 in their CSF, no further samples were analyzed for IL-23.

MS patients in the NIB cohort had higher levels of CXCL13 than patients with NIND, and again, this difference was driven by the RRMS subgroup. Similarly, OIND subjects had higher levels of CXCL13 in comparison to NIND patients. For IL-8, the only statistically significant difference resided in higher CSF values in OIND in comparison to NIND subjects.

Finally, a highly statistically significant correlation was observed for IL-12p40 and CXCL13 (R=0.585, P<10⁻⁴), but not with IL-8 (R=0.261, P=0.002).

### Correlations Between CSF Biomarkers of Intrathecal Inflammation and MRI CEL

CSF biomarker values were correlated against brain CEL in three ways. The first was the average number of gadolinium CEL on three consecutive monthly MRIs, which gave a measure of overall disease activity. The second was the number of CEL in the MRI closest to the LP (5.3+/−5.7 days for all patients), and the third was the number of CEL in the closest MRI that was performed prior (12.4+/−9.0 days for all patients) to the LP.

Out of the patients with the complete MRI dataset, 60% (37/62) of RRMS patients, 13% (4/32) of PPMS patients, 38% (3/8) of SPMS patients and 20% (2/10) of OIND patients had CEL. No patient in the CIS or NIND group had CEL lesions. Thus, only the all NIB, all MS, and RRMS groups were considered when assessing correlations between CSF proteins and CEL, since these are the only groups that had a substantial number of patients with CEL. Amongst correlations involving IL-12p40 and CXCL13, thirteen correlations between brain MRI measures of disease activity were significant with a P value of less than 0.05, and these are shown in Table 3. IL-8 did not significantly correlate with any brain MRI measure in any group. Since numerous correlations were made, a Bonferroni correction for multiple comparisons was used to derive a corrected P value.

IL-12p40 correlated better with the number of CEL than CXCL13, for all comparisons performed. The strongest correlations was observed when MRI preceded the LP(R=0.431, p<10⁻⁴ for all NIB cohort and R=0.465, p=0.008 for MS patients).

TABLE 1
Diagnostic and demographic data of the pilot (WMS) and confirmatory (NIB) cohorts

| | | | | | | | |
|---|---|---|---|---|---|---|---|
| RRMS | PPMS | SPMS | All MS | CIS | NIND | OIND | Total |

Pilot (WMS)

| | | | | | | | | | | | | | | | | |
|---|---|---|---|---|---|---|---|---|---|---|---|---|---|---|---|---|
| N (female/ | 25 | (22/3) | 1 | (0/1) | 2 | (0/2) | 28 | (22/6) | 10 | (9/4) | 27 | (21/6) | 7 | (6/1) | 72 | (58/14) |
| male) | | | | | | | | | | | | | | | | |
| Average Age | 37.1 | (8.1) | 56.7 | (—) | 51.6 | (2.8) | 38.9 | (9.4) | 43.9 | (12.4) | 44.4 | (11.2) | 45.2 | (11.5) | 42.3 | (10.9) |
| (SD) | | | | | | | | | | | | | | | | |
| Average EDSS | 1.7 | (1.1) | 2.5 | (—) | 4.3 | (3.2) | 1.9 | (1.4) | 1.2 | (1.1) | 0.8 | (1.2) | 1.8 | (0.6) | 1.5 | (1.3) |
| (SD) | | | | | | | | | | | | | | | | |
| Average | 91.4 | (7.0) | 91.0 | (—) | 77.0 | (12.7) | 90.3 | (8.1)^{a} | 95.7 | (5.3) | 97.1 | (5.3) | 88.7 | (7.3)^{a} | 93.0 | (7.5) |
| S-NRS | | | | | | | | | | | | | | | | |
| (SD) | | | | | | | | | | | | | | | | |
| Average | 1.3 | (1.1) | 1.2 | (—) | 1.3 | (0.4) | 1.3 | (1.1) | 1.3 | (1.4) | 0.5 | (0.1) | 0.5 | (0.1) | 0.9 | (0.9) |
| IgG Index | | | | | | | | | | | | | | | | |
| (SD) | | | | | | | | | | | | | | | | |

Confirmatory (NIB)

| | | | | | | | | | | | | | | | | |
|---|---|---|---|---|---|---|---|---|---|---|---|---|---|---|---|---|
| N (female/ | 66 | (39/27) | 33 | (16/17) | 8 | (4/4) | 107 | (59/48) | 9 | (5/4) | 33 | (27/6) | 18 | (6/12) | 167 | (97/70) |
| male) | | | | | | | | | | | | | | | | |
| Average Age | 29.5 | (10.8)^{b,c} | 52.8 | (7.0) | 52.6 | (13.3) | 48.0 | (11.8) | 41.3 | (13.4)^{c} | 48.1 | (9.7) | 41.5 | (13.5) | 45.1 | (11.6) |
| (SD) | | | | | | | | | | | | | | | | |
| Average EDSS | 1.7 | (1.4)^{b} | 5.1 | (1.8) | 5.5 | (1.7) | 3.0 | (2.3) | 1.0 | (1.1)^{b} | 2.6 | (2.2) | 2.4 | (2.0) | 2.8 | (2.2) |
| (SD) | | | | | | | | | | | | | | | | |
| Average | 92.0 | (9.4)^{b} | 67.8 | (16.1)^{d} | 71.0 | (12.7) | 82.5 | (16.8) | 96.0 | (0.9)^{b} | 90.5 | (12.3) | 80.0 | (14.4) | 84.1 | (16.1) |
| S-NRS | | | | | | | | | | | | | | | | |
| (SD) | | | | | | | | | | | | | | | | |
| Average | 1.4 | (0.9)^{d} | 0.9 | (0.6)^{d} | 0.7 | (0.3) | 1.0 | (0.7)^{a} | 0.7 | (0.3) | 0.5 | (0.1) | 0.8 | (0.6)^{a} | 8.9 | (0.7) |
| IgG Index | | | | | | | | | | | | | | | | |
| (SD) | | | | | | | | | | | | | | | | |

PPMS, primary progresive MS;
SPMS, secondary-progressive MS;
CIS clinically isolate syndrome;
NIND, non-inflammatory neurological diseases theadache, vascular-ischemic white matter changes, mitochandrial disorder, leukodystrophy, ALS, pseudotumor corebri, benign (asculation syndrome, and non-specific white matter lesions of enclear significance);
OIND, other inflammatory neurological diseases (CNS lupus, neurosacroid, Sjogren's syndrome, vasculitis, encepballtis, meningitis, Mashimoto's encephalitis, CNS lyme disease).
EDSS, expanded disability status value:
S-NRS, Scripps-neurological rating scale
^{a}P < 0.05 Dann's post-test (DPT) vs NIND on four groups.
^{b}P < 0.05 DPT vs. PPMS as six groups.
^{c}P < 0.05 DPT vs. SPMS on six groups.
^{d}P < 0.05 DPT vs. NIND on six groups.

TABLE 2
CSF protein concentrations in the pilot (WMS) and confirmatory (NIB) cohorts

| | | WMS | | | NIB | | |
|---|---|---|---|---|---|---|---|
| | | IL-12p46 | CXCL13 | IL-8 | IL-12p40 | CXCL13 | IL-8 |
| RRMS | Median | 4.3 pg/ml | 40.5 pg/ml | 31.4 pg/ml | 4.1 pg/ml | 122.6 pg/ml | 32.4 pg/ml |
| | (Range) | (2.1-99.7) | (25.0-145.9) | (22.8-250.0) | (2.3-36.3) | (62.5-955.7) | (19.5-117.2) |
| | % | 88% | 71% | 100% | 55% | 56% | 89% |
| | Detectable | (22/25) | (17/24) | (25/25) | (31/56) | (34/61) | (50/56) |
| PPMS | Median | 2.1 pg/ml | 25.6 pg/ml | 36.4 pg/ml | 2.3 pg/ml | 62.5 pg/ml | 35.1 pg/ml |
| | (Range) | (—) | (—) | (—) | (2.3-24.9) | (62.5-420.3) | (19.5-105.1) |
| | % | 0% | 0% | 100% | 35% | 34% | 92% |
| | Detectable | (0/1) | (0/1) | (1/1) | (9/26) | (10/29) | (24/26) |
| SPMS | Median | 4.7 pg/ml | 26.8 pg/ml | 37.2 pg/ml | 2.3 pg/ml | 62.5 pg/ml | 44.6 pg/ml |
| | (Range) | (2.1-4.7) | (25-28.5) | (36.9-37.6) | (2.3-2.9) | (62.5-142.1) | (25.9-65.4) |
| | % | 50% | 50% | 100% | 20% | 29% | 100% |
| | Detectable | (1/2) | (1/2) | (2/2) | (1/5) | (2/7) | (5/5) |
| All MS | Median | 4.2 pg/ml | 39.2 pg/ml | 36.7 pg/ml | 2.3 pg/ml | 62.5 pg/ml | 33.1 pg/ml |
| | (Range) | (2.1-55.7) | (25.0-145.9) | (22.8-250.0) | (2.3-36.3) | (62.5-955.7) | (19.5-117.2) |
| | % | 82% | 67% | 100% | 47% | 47% | 91% |
| | Detectable | (23/28) | (18/27) | (28/28) | (41/87) | (46/97) | (79/87) |
| CIS | Median | 2.2 pg/ml | 25.0 pg/ml | 30.3 pg/ml | 2.3 pg/ml | 62.5 pg/ml | 32.9 pg/ml |
| | (Range) | (2.1-59.0) | (25.0-109.2) | (16.5-51.0) | (2.3-2.7) | (62.5-158.4) | (19.9-64.2) |
| | % | 50% | 40% | 100% | 20% | 25% | 100% |
| | Detectable | (5/10) | (4/10) | (10/10) | (1/5) | (2/8) | (5/5) |
| NIND | Median | 2.1 pg/ml | 25.0 pg/ml | 22.6 pg/ml | 2.3 pg/ml | 62.5 pg/ml | 27.0 pg/ml |
| | (Range) | (2.1-2.1) | (25.0-151.6) | (16.1-250.0) | (2.3-14.0) | (62.5-613.9) | (19.5-53.1) |
| | % | 0% | 32% | 100% | 6% | 10% | 81% |
| | Detectable | (0/26) | (8/25) | (26/26) | (2/32) | (3/31) | (26/32) |
| OIND | Median | 2.1 pg/ml | 25.0 pg/ml | 38.2 pg/ml | 3.0 pg/ml | 173.2 pg/ml | 45.6 pg/ml |
| | (Range) | (2.1-3.05) | (25.0-120.8) | (22.8-45.9) | (2.3-36.3) | (62.5-626.7) | (24.0-113.3) |
| | % | 17% | 67% | 100% | 67% | 76% | 100% |
| | Detectable | (1/6) | (3/7) | (6/6) | (8/12) | (13/17) | (12/12) |

TABLE 3
Correlations of disease activity and CSF proteins in the confirmatory
(NIB) cohort

| | MRI Measure | | | | |
|---|---|---|---|---|---|
| Protein | of Disease Activity | Group | P* | r | N |
| IL-12p40 | Average number of CEL on 3 | All NIB | 0.039 | 0.296 | 99 |
| | Consecutive MRI | All MS | 0.322 | 0.271 | 69 |
| | MRI closest to LP | All NIB | 0.025 | 0.280 | 121 |
| | | All MS | 0.151 | 0.281 | 80 |
| | MRI closest, but prior to LP | All NIB | <10⁻⁴ | 0.431 | 82 |
| | | All MS | 0.008 | 0.465 | 51 |
| | | RRMS | 0.114 | 0.457 | 32 |
| CXCL13 | Average number of CEL on 3 | All NIB | 0.047 | 0.289 | 100 |
| | Consecutive MRI | All MS | 0.628 | 0.237 | 70 |
| | MRI closest to LP | All NIB | 0.156 | 0.219 | 131 |
| | | All MS | 0.533 | 0.220 | 87 |
| | MRI closest, but prior to LP | All NIB | 0.058 | 0.305 | 86 |
| | | All MS | 0.265 | 0.315 | 54 |

*P value after Bonferroni correction (n = 13).

### DISCUSSION

While there are many studies that reported elevated levels of different cytokines in the CSF of patients with MS and controls, few of them are consistently reproduced. In a pilot study utilizing 10 fold-concentrated CSF and measuring wide array of cytokines (IL-6, IL-7, IL-8, IL-10, IL-12p40, IL-12p70, IL-17, IL-21, IL-23, IFN-γ, TNF-α, lymphotoxin-α, VEGF, oncostatin M, granzyme B, CX3CL1) with a highly sensitive cytometric bead array assay (Bielekova et al., Neurology 77(21):1877-1886, 2011), it was possible to consistently detect only IL-12p40 and IL-8 in untreated MS patients that had active intrathecal inflammation. Technical differences in sample collection, processing, and assay measurements may explain the differences between this data and those that report a more abundant cytokine profile in the CSF of MS patients. The present study focused on the detection of those soluble factors that have been released into the CSF in vivo, by eliminating release of cell-derived soluble factors after CSF collection. This was achieved by putting CSF samples on ice immediately after collection; spinning the CSF within 15 minutes of collection to remove cells and cryopreserving only cell-free supernatant. Second, only the linear part of the standard curve was used to derive results, which ensures that proteins are detected well above the noise of each assay. Third, in order to assure consistent detection within the linear part of standard curves, CSF was concentrated.

The strength of the present study resides in the analysis of two cohorts of untreated MS patients with embedded inflammatory and non-inflammatory neurological controls, prospectively acquired by the same investigators. Further, all samples were processed using identical standardized procedures and were evaluated on coded samples in a blinded fashion, eliminating non-biological differences that may occur due to different methods of CSF collection and storage. Furthermore, the preliminary results were validated in a large independent cohort, using deliberately more than one detection assay, thus assuring wide clinical applicability of the biomarkers. Finally, the relationship of detected CSF biomarkers with validated radiological markers of MS disease activity was assessed. The results demonstrate that both IL-12p40 and CXCL13 are useful measures of intrathecal inflammation that correlate with MS disease activity.

The present disclosure is the first to report low detectable levels (less than 20 pg/ml on average) of CSF IL-12p40 (but not IL-12p70 or IL-23), that are higher in MS, RRMS and OIND groups than in a group of patients with NIND. When human monocytes or macrophages are cultured in vitro, IL-12p40 is produced in greater abundance than IL-12p70 (>100 fold excess) and IL-23 (>10 fold excess), even if multiple different stimuli are utilized (Dobreva et al., Cytokine 43:76-82, 2008). This is fully compatible with the current data where IL-12p70 or IL-23 proteins could not be identified in the CSF, even in those patients in whom the highest levels of IL-12p40 were detected. It is believed that if cytokine release from cells is prevented, then in MS patients CSF levels of IL-12p70 and IL-23 fall below detectable thresholds of currently available assays. In fact, the low amount of IL-12p40 in the CSF has probably prevented others from showing that IL-12p40 is increased in MS in instances where the CSF was not concentrated (Braitch et al., Arch Neurol 65:633-635, 2008). Interestingly, IL-12p40 not only forms a part of IL-12p70 and IL-23, but is also biologically active as a homodimer (IL-12p80), at least in mice (Holscher, Med Microbiol Immunol 193:1-17, 2004; Cooper and Khader, Trends Immunol 28:33-38, 2007). Both antagonistic properties due to blockade of IL-12/IL-23 signaling pathway and inherently agonistic properties, such as inhibition of T regulatory cells (Brahmachari and Pahan, J Immunol 183:2045-2058, 2009) or induction of lymphotoxin-a (Jana et al., Glia 57:1553-1565, 2009) have been assigned to IL-12p80. Patients with SPMS were found to have a significantly higher production of IL-12p40 by their peripheral blood mononuclear cells (PBMC) (Soldan et al., J Neuroimmunol 146:209-215, 2004), and interferon-13 suppresses IL-12p40 production (Alexander et al., Mult Scler 16:801-809, 2010).

After unblinding, only one NIND patient, NIB 135, had higher CSF IL-12p40 (14.04 pg/ml) than the selected cut-off of 2.4 pg/ml. Although NIB 135, who carried a diagnosis of SLE, was classified to the NIND group by the clinical staff based on her lack of CEL, retrospective review indicated that NIB 135 had CSF pleocytosis (7 white blood cells per microliter) and CSF specific OCB, both of which indicate intrathecal inflammation. Therefore, NIB 135 was found to have been misclassified. Nevertheless, NIB 135 was kept in the NIND category throughout all analyses.

The current disclosure is also the first to show that IL-12p40 correlates with brain CEL, in fact better than CXCL13. A plausible hypothesis why the strongest correlation was observed for CEL detected on the MRI that preceded the LP is that the opening of the blood brain barrier (BBB) recruited a large number of blood-derived monocytes that became activated and released IL-12p40 after myelin phagocytosis. This would indicate that IL-12p40 itself does not participate in the events leading to BBB opening, either directly or indirectly. The fact that systemic administration of IL-12p40-targeting therapies does not abrogate development of CEL (Segal et al., Lancet Neurol 7:796-804, 2008) supports the hypothesis about lack of causative relationship between IL-12p40 and BBB opening in MS.

This study was able to reproduce findings that CXCL13 is increased in patients with MS and that CXCL13 correlates with brain MRI measures of disease activity (Mellergard et al., Mult Scler 16:208-217, 2010). Some reports have claimed that IL-8 is increased in the CSF of patients with MS (Ishizu T et al., Brain 128:988-1002, 2005; Franciotta et al., J Neurol Sci 247:202-207, 2006), while others have shown that there is no difference in IL-8 levels between MS patients and controls (Saruhan-Direskeneli et al., J Neuroimmunol 145:127-134, 2003; McDonald et al., Ann Neurol 50:121-7, 2001; Dobreva et al., Cytokine 43:76-82, 2008). The failure of IL-8 to correlate with MRI measures of disease suggests that IL-8 might not be a direct cause or result of specific CNS disease pathology in MS. This conclusion is supported by the data that daclizumab treatment, which results in profound inhibition of CEL, inhibits CSF levels of IL-12p40, but not of IL-8 (Bielekova et al., Neurology 77(21):1877-1886, 2011).

The fact that 23.5% (8/34) of patients with progressive MS had elevated CSF levels of IL-12p40 (in comparison to 0/57 properly classified NIND patients) indicates that CSF analysis of IL-12p40 (alone or in combination with CXCL13) can identify those patients with progressive MS that have active intrathecal inflammatory process amenable to immunomodulatory treatments that can bypass the closed BBB, such as IT rituximab. Similarly, these biomarkers find clinical utility in aiding and monitoring therapeutic decisions in patients with OIND. The results of the present study indicate that evaluation of CSF IL-12p40 can be used as a biomarker for studies of intrathecal inflammation in MS and other neuroimmunological disorders of the CNS.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. A method of treating a subject having multiple sclerosis (MS), comprising selecting a subject with MS and administering to the subject a therapeutically effective amount of rituximab into the cerebral spinal fluid (CSF), thereby treating the subject with MS.

2. The method of claim 1, wherein administering rituximab into the CSF comprises administering rituximab intrathecally.

3. The method of claim 2, wherein intrathecal administration comprises administration by lumbar puncture and infusion into the intrathecal space of the spinal cord.

4. The method of claim 2, wherein the therapeutically effective amount of rituximab administered intrathecally is about 10 mg to about 50 mg per dose.

5. (canceled)

6. The method of claim 2, wherein the therapeutically effective amount of rituximab administered intrathecally is about 25 mg per dose.

7. The method of claim 1, wherein the subject is administered a single dose of rituximab intrathecally.

8. The method of claim 1, wherein the subject is administered multiple doses of rituximab intrathecally.

9. The method of claim 8, wherein the subject is administered two or three doses of rituximab intrathecally.

10. The method of claim 9, wherein the subject is administered a first, second and third dose of about 25 mg rituximab intrathecally.

11-12. (canceled)

13. The method of claim 1, wherein the subject is further administered a therapeutically effective amount of rituximab intravenously.

14. The method of claim 13, wherein the therapeutically effective amount of rituximab administered intravenously is about 150 mg to about 300 mg per dose.

15. (canceled)

16. The method of claim 14, wherein the therapeutically effective amount of rituximab administered intravenously is about 200 mg per dose.

17. The method of claim 13, wherein the subject is administered a single dose of rituximab intravenously.

18. The method of claim 13, wherein the subject is administered multiple doses of rituximab intravenously.

19. The method of claim 18, wherein the subject is administered two doses of rituximab intravenously.

20. The method of claim 19, wherein the subject is administered two doses of about 200 mg rituximab intravenously.

21-22. (canceled)

23. The method of claim 1, wherein the subject has secondary progressive MS.

24. A method of treating a subject having multiple sclerosis (MS), comprising:
selecting a subject with secondary progressive MS (SPMS);
administering to the subject a first dose of 25 mg rituximab into the CSF and a first intravenous dose of 200 mg rituximab, wherein the first dose of rituximab into the CSF and the first intravenous dose of rituximab are administered about 2-24 hours apart;
administering to the subject a second intravenous dose of 200 mg rituximab about two weeks following the first intravenous dose;
administering to the subject a second dose of 25 mg rituximab into the CSF about six weeks after the first dose into the CSF; and
administering to the subject a third dose of 25 mg rituximab into the CSF about 12 months following the first dose into the CSF, thereby treating the subject having MS.

25. The method of claim 24, wherein the first dose of rituximab into the CSF is administered about 4 hours prior to the first intravenous dose of rituximab.

26. The method of claim 24 or claim 25, wherein administering rituximab into the CSF comprises administering rituximab intrathecally.

27. The method of claim 26, wherein intrathecal administration comprises administration by lumbar puncture and infusion into the intrathecal space of the spinal cord.

28. (canceled)

29. The method of claim 1, wherein selecting the subject with MS comprises measuring the level of IL-12p40, CXCL13 or both in the CSF of the subject, and selecting the subject if expression of IL-12p40, CXCL13, or both is increased in the CSF relative to a control.

30-31. (canceled)

32. A method of evaluating the effectiveness of a therapy in a subject having MS, comprising measuring the level of IL-12p40, CXCL13, or both in the CSF of the patient before and after treatment with the therapy, wherein a decrease in the level of IL-12p40, CXCL13, or both after treatment compared to before treatment indicates the therapy is effective.

33. A method of selecting a patient having progressive MS as a candidate for treatment with an intrathecal immunomodulatory therapy, or a method of identifying a progressive MS patient as having meningeal inflammation, comprising measuring the level of IL-12p40, CXCL13, or both in the CSF of the patient, wherein an increase in the level of IL-12p40, CXCL13, or both relative to a control, indicates the subject is a candidate for treatment with an intrathecal immunomodulatory therapy, or indicates that the MS patient has meningeal inflammation.

34-43. (canceled)
